(19) United States
(12) Patent Application Publication
(10) Pub. No.: US 2017/0028150 A1
(43) Pub. Date: Feb. 2, 2017

(54) CONVERSION MASK

(71) Applicant: Linda McNulty, Fairlawn, OH (US)

(72) Inventor: Linda McNulty, Fairlawn, OH (US)

(21) Appl. No.: 15/225,140

(22) Filed: Aug. 1, 2016

Related U.S. Application Data
(60) Provisional application No. 62/199,331, filed on Jul. 31, 2015.

(51) Int. Cl. A61M 16/06

(52) U.S. Cl. CPC A61M 16/0672; A61M 16/0683; A61M 2230/205

(57) ABSTRACT

One or more embodiments of the present invention provide a conversion mask that is quick, safe and cost effective and a method for increasing oxygen saturation and/or arresting a subsequent decline in oxygen saturation, which does not unnecessarily increase costs, require a bulky and uncomfortable mask or disrupt the flow of oxygen and/or care being provided to the patient. In one or more embodiments, the conversion mask has a thin, lightweight non latex plastic shield or tent-like mask designed to convert a nasal cannula to a full oxygen capturing mask. The conversion mask serves to capture oxygen escaping from nasal cannula effectively redirecting it back to the mouth and nose, thereby improving oxygenation and/or arresting a decline in oxygenation of patients, particular those under conscious sedation, deep Propofol anesthesia or post general anesthesia, with low oxygen saturation levels until they can be transferred to next level care.

### CROSS REFERENCE TO RELATED APPLICATIONS

This application claims the benefit of U.S. provisional patent application Ser. No. 62/199,331 entitled “Conversion Mask,” filed Jul. 31, 2015, and incorporated herein by reference in its entirety.

### FIELD OF THE INVENTION

One or more embodiments of the present invention relates to a device and method for providing oxygen to a patient. In certain embodiments, the present invention is directed to a safe and cost effective device and method for increasing oxygen saturation and/or arresting a subsequent decline in oxygen saturation that does not unnecessarily increase costs, require a bulky and uncomfortable mask or disrupt the flow of oxygen and/or care being provided to the patient.

### BACKGROUND OF THE INVENTION

It is well known that prolonged oxygen deprivation can lead to brain damage and, eventually death. Blood oxygen saturation is a measurement of the amount of oxygen being transported from the lungs to the various cells, tissues and organs of the body. It is also known that procedures like conscious sedation, deep Propofol anesthesia or general anesthesia suppress normal respiration, requiring various degrees of supplemental oxygenation for the patient during and immediately after these procedures to prevent dangerous oxygen deprivation. Supplemental oxygenation is not limited to these types of patients, however, and is also be commonly used for patients with compromised lung and breathing function.

In many clinical settings, a nasal cannula is used to provide supplemental oxygenation to patients with low blood oxygen saturation, particularly during and/or after conscious sedation, deep Propofol anesthesia or post general anesthesia, to increase oxygen saturation until the patient can be transferred to next level care. Blood oxygen saturation is a measurement of the amount of oxygen being transported from the lungs to the various cells, tissues and organs of the body. In some cases, however, the nasal cannula fails to provide the oxygenation necessary to sufficiently increase blood oxygen saturation or to arrest a subsequent decline in blood oxygen saturation, so that the patient can be transferred to next level care. This failure, may be the result of a variety of factors including, by way of example, nasal blockage or chronic breathing issues.

In these cases, the nasal cannula is often removed and discarded and a full oxygen capturing mask used to provide to oxygenation through both the nose and mouth. While a full oxygen capturing mask is generally effective in increasing oxygen saturation and/or arresting a subsequent decline in oxygen saturation, these masks are relatively expensive, bulky, uncomfortable, and difficult to put on an unconscious or heavily sedated patient. More importantly, changing from the nasal cannula to the full oxygen capturing mask results in a disruption both to the flow of oxygen to the patient and the flow of care being provided to the patient. And when the oxygen saturation level has been improved and/or stabilized to the point where the patient can be transferred to next level care, these masks are removed and discarded and a second nasal cannula installed to provide ongoing oxygenation, further increasing the costs to the patient.

What is needed in the art is a is quick, safe and cost effective device and method for increasing oxygen saturation and/or arresting a subsequent decline in oxygen saturation that does not unnecessarily increase costs, require a bulky and uncomfortable mask or disrupt the flow of oxygen and/or care being provided to the patient.

### SUMMARY OF THE INVENTION

One or more embodiments, the present invention provides a conversion mask that is quick, safe and cost effective device and method for increasing oxygen saturation and/or arresting a subsequent decline in oxygen saturation that does not unnecessarily increase costs, require a bulky and uncomfortable mask or disrupt the flow of oxygen and/or care being provided to the patient. In one or more embodiments, the present invention relates to a thin, lightweight non latex plastic shield or tent like mask designed to convert a nasal cannula to a full oxygen capturing mask to capture oxygen escaping from nasal cannula with resultant decline in oxygenation of patients, awake, conscious sedation, deep propofol anesthesia or post general anesthesia patients with low oxygen saturation to increase it till transferred to next level care. It is designed for temporary use and only in conjunction with nasal cannula.

One or more embodiments of the present invention, the conversion mask of the present invention is very inexpensive and may quickly and easily be applied to the patients face without significantly disturbing the flow of oxygen or the other care being provided to the patient. In these embodiments, the nasal cannula stays in place throughout as the source of oxygen and to measure levels of carbon dioxide, eliminating the need to switch from a nasal cannula to conventional expensive face mask, and then back to nasal cannula. In various embodiments, the conversion mask of the present invention can be used in outpatient facilities, post surgery areas, transporting patients, emergency rooms and EMS squads. In some other embodiments, the conversion mask of the present invention can also be used as a protective shield for combative spitting patients.

A series of perforations and small holes allows air exchange and thus the conversion mask of the various embodiments of the present invention will stick to the face of or accidentally suffocate the patient. It is loose fitted and only an adjunct to nasal oxygen when the oxygen level is low and/or declining and brings the level up by providing a tent like effect and traps all oxygen near the mouth and nose. When the oxygen level returns to an acceptable level the conversion mask can be removed and the patient returned to the use of nasal oxygen alone.

### BRIEF DESCRIPTION OF THE DRAWINGS

For a more complete understanding of the features and advantages of the present invention, reference is now made to the detailed description of the invention along with the accompanying figures in which:

FIG. 1 is a side view of a conversion mask according to one or more embodiments of the present invention;

FIG. 2 is a front view of a conversion mask according to one or more embodiments of the present invention;

FIG. 3 is a front view a mannequin fitted with a nasal cannula to demonstrate how nasal cannula conversion mask may be worn by a patient;

FIG. 4 is a front view of a conversion mask according to one or more embodiments of the present invention placed on a mannequin fitted with a nasal cannula to demonstrate how the conversion mask may be worn by a patient; and

FIG. 5 is a side view of a conversion mask according to one or more embodiments of the present invention placed on a mannequin fitted with a nasal cannula to demonstrate how the conversion mask may be worn by a patient.

### DETAILED DESCRIPTION OF THE ILLUSTRATIVE EMBODIMENTS

One or more embodiments of the present invention provides a conversion mask that is quick, safe and cost effective and a method for increasing oxygen saturation and/or arresting a subsequent decline in oxygen saturation, which do not unnecessarily increase costs, require a bulky and uncomfortable mask or disrupt the flow of oxygen and/or care being provided to the patient. In one or more embodiments, the present invention relates to a conversion mask having a thin, lightweight non-latex plastic shield or tent-like mask designed to convert a nasal cannula to a full oxygen capturing mask. The conversion mask serves to capture oxygen escaping from nasal cannula due to a nasal blockage or other factors, effectively redirecting it back to the mouth and nose, thereby improving oxygenation and/or arresting a decline in oxygenation of patients, particular those under conscious sedation, deep propofol anesthesia or post general anesthesia, with low oxygen saturation levels, until they can be transferred to next level care. The conversion mask of the present invention is designed for temporary use and only in conjunction with a nasal cannula.

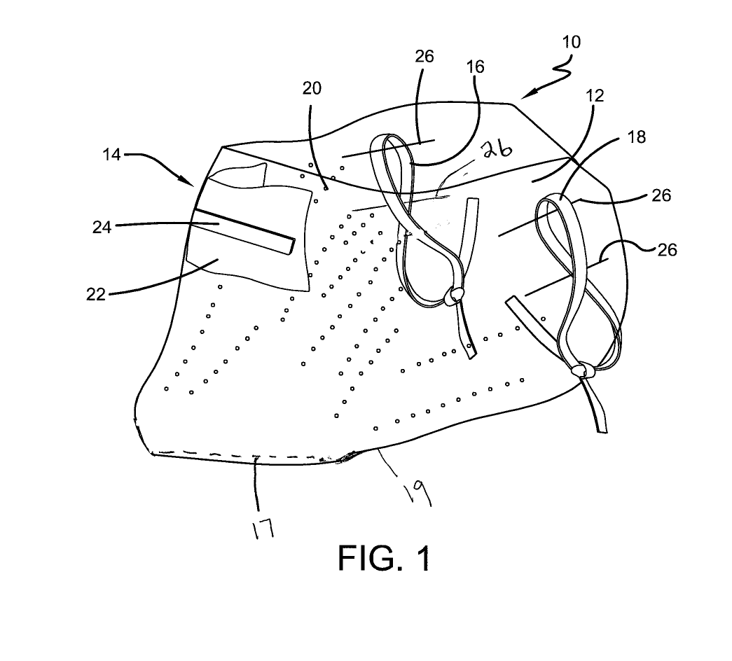

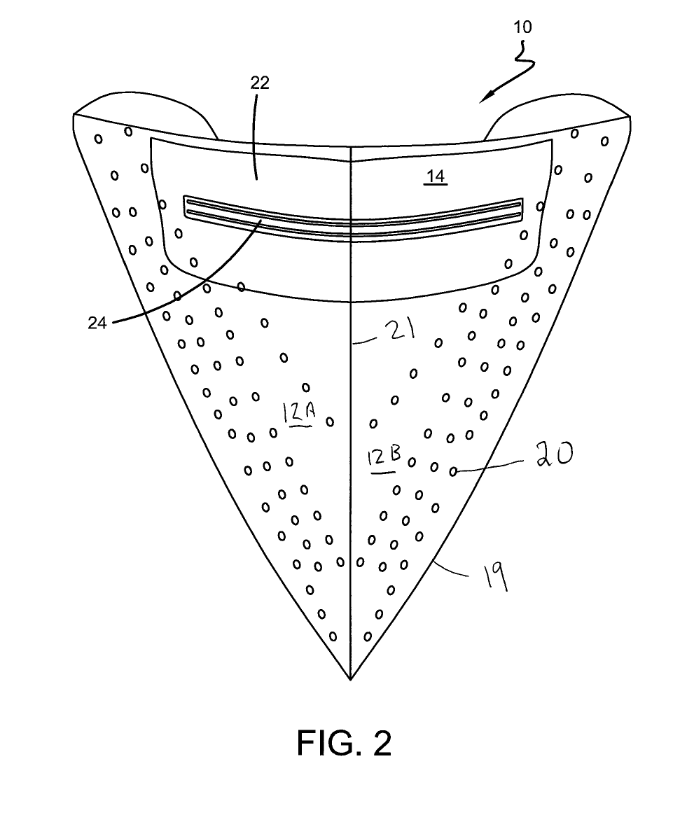

Referring now to FIGS. 1 and 2, a conversion mask is shown, generally indicated by the numeral 10. In one or embodiments, conversion mask 10 includes a plastic shield/mask 12, a nose pad 14, first and second ear straps 16, 18, and perforations 20. Plastic shield/mask 12 may be made from any flexible material having sufficient stiffness to prevent being sucked onto the face or into the nose or mouth of the patient. One of ordinary skill in the art will be able to select a suitable material and material thickness for the plastic shield/mask 12 without under experimentation. Suitable materials may include, without limitation, polypropylene, vinyl, Mylar® or polyester. Suitable material thicknesses will of course depend upon the material chosen, but may be from about 0.1 mm to about 1.5 mm. In some embodiments, plastic shield/mask 12 may be translucent. Further, plastic shield/mask 12 should be non-allergenic and made of a material that may safely come into contact with the skin of the patient.

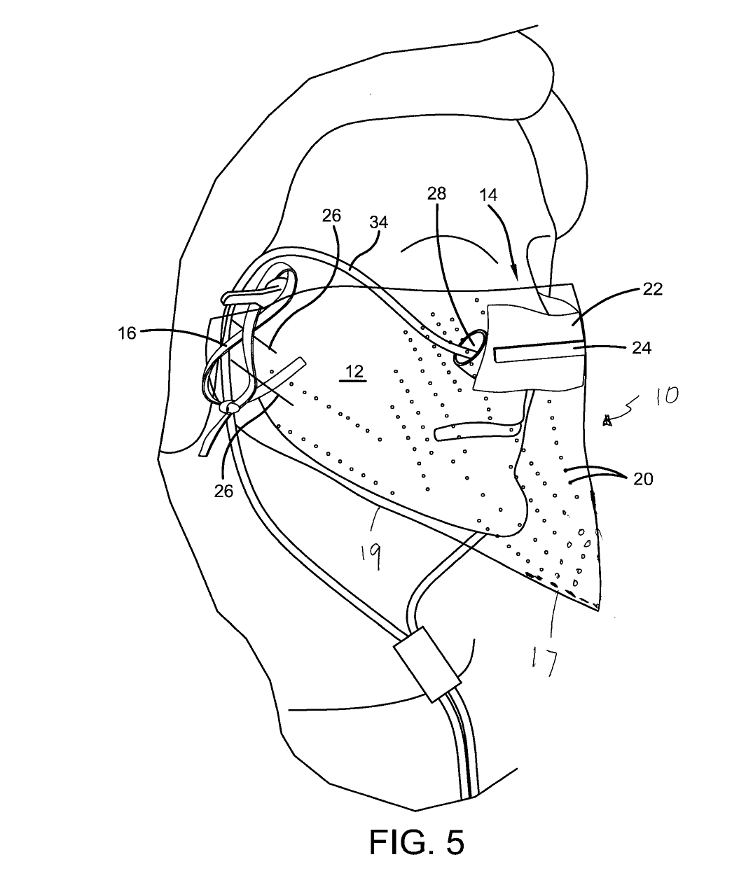

As can be seen in FIGS. 1, 4-5, conversion mask is configured to fit over the face of the patient, running from the top of the nose to below (and preferably under) the chin and from ear to ear. In one or more embodiments, plastic shield/mask 12 may be formed of a single piece of material molded to fit over the face as described. In some other embodiments, plastic shield/mask 12 may be formed of a single sheet of material that is folded along a center line 21, corresponding generally to the midline of the patient's face running from the nose to the center of the chin of the patient's face. In these embodiments, a seam 17 may be formed along the bottom edge 19 of plastic shield/mask 12 between the two sides 12A, 12B of plastic shield/mask 12, running from where they meet the fold at center line 21 back toward the face of the patient (see FIG. 2), thereby forming a pocket for the patient's chin (see FIGS. 1 and 5). In some other embodiments, a second piece of material may be added between the two sides of plastic shield/mask 12 and extending under the chin of the patient.

Plastic shield/mask 12 further comprises a series of perforations 20 of varying sizes intended to permit additional air flow in and out of the conversion mask 10. The number and size of perforations 20 is not particularly limited except to the extent that they are large and/or numerous enough will to allow enough air flow that plastic shield/mask 12 does not stick to the face of or accidentally suffocate the patient then they breath, but not so numerous or large as to render plastic shield/mask 12 incapable of collecting a substantial portion of the oxygen gas escaping from the nasal cannula.

In one or more embodiment, perforations 20 may mechanically punched into plastic shield/mask 12, but the invention is not so limited and perforations 20 may be formed by any conventional means. In various embodiments, perforations 20 may have a diameter of from 0.1 mm to about 3.0 mm. In some of these embodiments, perforations 20 may have a diameter of 0.3 mm or more, in other embodiments 0.5 mm or more, in other embodiments 0.8 mm or more, in other embodiments 1.0 mm or more, in other embodiments 1.3 mm or more, in other embodiments 1.5 mm or more. In some of these embodiments, perforations 20 may have a diameter of 2.5 mm or less, in other embodiments 2.3 mm or less, in other embodiments 2.0 mm or less, in other embodiments 1.8 mm or less, in other embodiments 1.5 mm or less, in other embodiments 1.0 mm or less.

In some embodiments, perforations 20 may be oriented in rows as shown in FIGS. 1-2, 4-5, but this need not be the case. In some other embodiments, perforations 20 may be located randomly, spaced at regular intervals, or concentrated in certain locations, such as adjacent to the nose or along the jaw line, to name just a few possible configurations.

Nose pad 14 sits over the nose of the patient and is secured to plastic shield/mask 12. In some embodiments, nose pad 14 further comprises a pad portion 22 and a moldable portion 24 capable of being bent to generally conform to the shape of the nose of the patient. In some embodiments, pad portion 22 is made of a soft rubberlike cushioned material that is non-allergenic and made of a material that may safely come into contact with the skin of the patient. Suitable materials may include, without limitation, foam rubber or cloth padding. In some embodiments, moldable portion 24 may be made from any material sufficiently malleable to permit manual shaping of the moldable portion 24 to generally conform to the shape of the top of the patient's nose. Suitable materials may include, without limitation, coated metal wire. In some embodiments, moldable portion 24 may be located between pad portion 22 and plastic shield/mask 12. In some other embodiments, moldable portion 24 may be located on the outside surface of the plastic shield/mask 12 such that the plastic shield/mask 12 is located between the moldable portion 24 and the pad portion 22.

First and second ear straps 16, 18 are secured to the areas of the plastic shield/mask 12 adjacent to the ears of the patient. First and second ear straps 16, 18 may be made of any material that is non-allergenic and may safely come into contact with the skin of the patient, but are preferably elastic bands. First and second ear straps 16, 18 may be secured to the plastic shield/mask 12 by any suitable means. In some embodiments, the first and second ear straps 16, 18 may be secured to the plastic shield/mask 12 through one or more holes 26 cut into the plastic shield/mask 12.

In some embodiments, conversion mask 10 may further include a flap cut through the plastic shield/mask 12 adjacent to permit medical instruments, such as an endoscope, to be inserted through conversion mask 10.

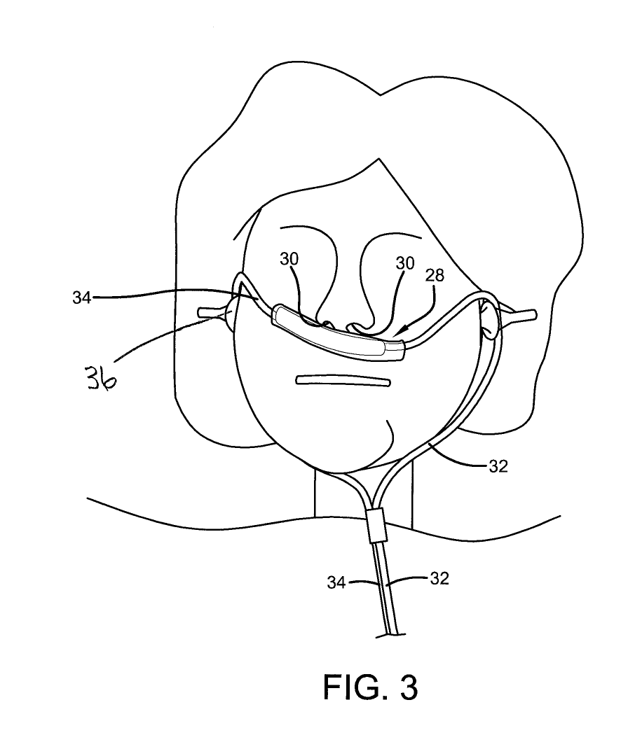

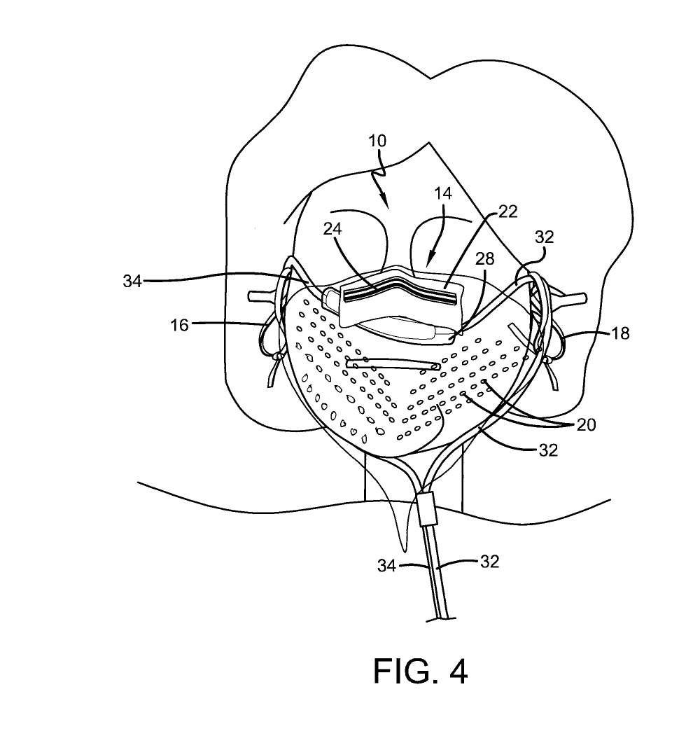

Turning now to FIGS. 3, which shows a mannequin fitted with a nasal cannula to demonstrate how nasal cannula 28 conversion mask may be worn by a patient and FIGS. 4 and 5, which show a conversion mask according to one or more embodiments of the present invention placed on a mannequin fitted with a nasal cannula to demonstrate how the conversion mask may be worn by a patient. As can be seen from FIGS. 4 and 5, conversion mask 10 fits over the face of the patient, covering both the patient's nose and mouth, and is secured to the patient's face by the first and second ear straps 16, 18. In some embodiments, to the plastic shield/mask 12 extends below the patient's chin.

Conversion mask 10 fits loosely against the face of the patient and is not intended to create a fluid seal. Accordingly, it should be apparent that conversion mask 10 is not intended for positive pressure ventilation or resuscitation.

In other embodiments, the present invention is directed to a method of supplying oxygen to a patient, particularly after during and/or after conscious sedation, deep Propofol anesthesia or post general anesthesia, using the conversion mask described above. It should be appreciated that these patients will have oxygen saturations levels that are lower than would be desired or, at best, at the low end of a range of adequate oxygen saturations levels. It is expected, or at least hoped, in these cases that the oxygen supplied to the patient's nostrils 30 by the nasal cannula 28 will be sufficient to bring the oxygen saturations levels up to a desired level or at the very least stabilize them until the patient moves to the next level of care. As set forth above, however, in some cases the oxygen provided to the nasal cannula 28 through oxygen supply tube 32 fails to enter through the patient's nasal passages and is ordinarily lost into the ambient air. The failure of the oxygen to enter through the patient's nasal passages may be the result of a variety of factors including, by way of example, nasal blockage or chronic breathing issues. As should be apparent, oxygen lost in this manner is not utilized by the patient and does not serve to improve or stabilize the patient's oxygen saturations levels.

Accordingly, in one or more of these embodiments, a patient is first fitted with a nasal cannula 28 configured to provide oxygen gas to the patient as shown in FIG. 3. In these embodiments, the nasal cannula 28 is sized to fit within the nostrils 30 of the patient and is connected to an oxygen supply tube 32 configured to bring oxygen gas to one or both of the patient's nostrils 30. In some embodiments, a second tube 34 configured to take some of the gasses exhaled by the patient to a carbon dioxide sensor (not shown).

Next, the oxygen saturation level in the patient's blood is monitored, continuously or at regular intervals using any one of various methods known in the art for that purpose, and the patient watched for signs of low oxygen saturation. In some embodiments, the patient is connected to a conventional electronic sensor configured to measure the oxygen saturation in the patient's blood (not shown) and the oxygen saturation levels are monitored continuously or at regular intervals.

If the measured oxygen saturation level drops or does not increase at a desired rate, or if it otherwise appears that the patient is not getting enough oxygen, the conversion mask 10 is fitted over the face of the patient and secured to the patient's ears 36 by first and second ear straps 16, 18, as shown in FIGS. 4 and 5. As should be apparent, there is no need to move, adjust, or replace nasal cannula 28 in order to fit conversion mask 10 to the face of the patient, as it lays over the top of nasal cannula 28, oxygen supply tube 32, and second tube 34 and does not interfere with their operation. Nose pad 14 is situated over the top of the nose and does not interfere with nasal cannula 28. While not particularly limited, oxygen supply tube 32 and second tube 34 run out from under conversion mask 10 along the sides of the patient's face, either above first and second ear straps 16, 18, as shown in FIGS. 4, 5, or below.

As set forth above, conversion mask 10 is loose fitted and brings the blood oxygen saturation level up by providing a tent like effect and traps all oxygen near the mouth and nose of the patient, thereby increasing the amount of oxygen absorbed by the patient. In these embodiments, the patient's oxygen saturation levels continue to be monitored, and once the oxygen saturation levels reach a predetermined desired level and appear to be stable, the conversion mask may be removed.

The conversion mask and related methods according to various embodiments of the present invention prevent many benefits over similar devices and methods known in the art. First, the conversion masks of the present invention are extremely inexpensive to make relative to the conventional full oxygen capturing mask, are lightweight, and easy to pack, ship and/or store. Convention mask 10 is much less bulky and uncomfortable that conventional full oxygen capturing mask. Moreover, the methods describe above require only one nasal cannula and a conversion mask, rather than two nasal cannula and conventional full oxygen capturing mask required by prior art methods. Another advantage, as set forth above, is that the conversion mask of the present invention can be quickly and easily applied to the patient's face without significantly disturbing the flow of oxygen or the other care being provided to the patient. The nasal cannula stays in place throughout as the source of oxygen and to measure levels of carbon dioxide, eliminating the need to switch from a nasal cannula to conventional expensive face mask, and then back to nasal cannula. In various embodiments, the conversion mask of the present invention can be used in outpatient facilities, post-surgery areas, transporting patients, emergency rooms and/or EMS squads. In some other embodiments, the conversion mask of the present invention can also be used as a protective shield for combative spitting patients.

In light of the foregoing, it should be appreciated that the present invention significantly advances the art by providing a conversion mask that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

What is claimed is:

1. An quick, safe and cost effective apparatus for use with a nasal cannula for increasing oxygen saturation and/or arresting a subsequent decline in oxygen saturation comprising:
a thin plastic shield/mask having a top, sized to fit over the nose of a patient, a bottom extending below the mouth of said patient, a first side extending from the mouth and nose of said patient along a first side of the head of a patient, and a second side extending from the mouth and nose of said patient along a second side of the head of said patient, said thin plastic shield/mask further comprising a plurality of perforations;
a padded nose piece;
a first ear strap secured to said first side of said thin plastic shield/mask; and
a second ear strap secured to said first second of said thin plastic shield/mask.

2. The apparatus of claim 1 wherein said first side further comprises a first bottom edge and said second side further comprises a second bottom edge, and a portion of said first and second bottom edges under or are joined to form a pocket.

3. The apparatus of claim 1 wherein said plurality of perforations have a diameter of from about 0.3 mm to about 1.5 mm.

4. The apparatus of claim 1 wherein said thin plastic shield/mask is made from a materials selected from the group consisting of, polypropylene, vinyl, Mylar®, polyester, and combinations thereof.

5. The apparatus of claim 1 wherein said thin plastic shield/mask is translucent.

6. The apparatus of claim 1 further comprising one or more openings sized to allow medical instruments to be inserted through said plastic shield/mask.

7. A method for supplying oxygen to a patient using the apparatus of claim 1 comprising:
A. fitting a patient with a nasal cannula;
B. monitoring the blood oxygen concentration of said patient;
C. preparing a conversion mask comprising a thin plastic shield/mask having a top, sized to fit over the nose of a patient, a bottom extending below the mouth of said patient, a first side extending from the mouth and nose of said patient along a first side of the head of a patient, and a second side extending from the mouth and nose of said patient along a second side of the head of said patient, said thin plastic shield/mask further comprising a plurality of perforations, a padded nose piece, a first ear strap secured to said first side of said thin plastic shield/mask; and a second ear strap secured to said first second of said thin plastic shield/mask;
D. determine whether the patient's blood oxygen concentration is below a predetermined level or is decreasing, and if so, fitting said conversion mask over the face of said patient and said nasal cannula and then securing said conversion mask to the ears of said patient using said first and second ear straps;
E. monitoring the blood oxygen concentration of said patient; and
F. determining whether the patient's blood oxygen concentration is at or above a predetermined level and if so, removing said conversion mask.

8. The method of claim 7 wherein said blood oxygen concentration of said patient is monitored continuously or at regular intervals using an electroinic blood oxygen concentration sensor.
